(19) United States
(12) Patent Application Publication
(10) Pub. No.: US 2024/0132951 A1
(43) Pub. Date: Apr. 25, 2024

(54) ANALYSIS METHOD OF BASE SEQUENCE AND GENE ANALYZER

(71) Applicant: Hitachi High-Tech Corporation, Tokyo (JP)

(72) Inventor: Toru YOKOYAMA, Tokyo (JP)

(73) Assignee: Hitachi High-Tech Corporation, Tokyo (JP)

(21) Appl. No.: 18/555,972

(22) PCT Filed: May 17, 2021
(86) PCT No.: PCT/JP2021/018618

§ 371 Date: Oct. 18, 2023

(51) Int. Cl. C12Q 1/6869

(52) U.S. Cl. CPC C12Q 1/6869; C12Q 1/6806

(57) ABSTRACT

A gene analyzer that analyzes a base sequence of a sample manages an observation environment and mobility correction amount data for correcting a position in a time direction of time-series data of signal intensities of a plurality of bases, and executes the following processes including: a process of scaling the mobility correction amount data associated with the observation environment different from a first observation environment to generate default mobility correction amount data when the gene analyzer receives time-series data of signal intensities of a plurality of bases acquired by electrophoresing the sample in the first observation environment; a process of correcting the position in the time direction of the time-series data of the signal intensities of the plurality of bases using an optimization algorithm; and a process of identifying the base sequence of the sample using the corrected time-series data of the signal intensities of the plurality of bases.

### TECHNICAL FIELD

The present invention relates to a gene analyzer that analyzes a base sequence of a sample using electrophoresis and a method thereof.

### BACKGROUND ART

In analysis of a base sequence of DNA using a capillary electrophoresis device, a mobility correction process of correcting a difference in migration speed between bases is executed (for example, refer to NPL 1). Regarding the mobility correction process, for example, techniques described in PTLs 1 and 2 are known.

PTL 1 describes “detection data of four types of bases is obtained (S1), other detection data are shifted with respect to detection data of a reference base as reference detection data (S2), a total area of peak waveforms is calculated (S3), the shift amount of the other detection data in which the total area of the peak waveforms is the maximum is obtained using a function for mobility correction as a linear shift (S4), position information of the detection data is corrected (S5), a temporary base sequence is determined (S6), using the detection data of the reference base as the reference detection data, a target peak before and after which reference peaks of the reference detection data are present is selected from the other detection data (S7), peak-to-peak intervals between the target peak and each of the reference peaks is obtained (S8), the shift amount of the target peak is calculated such that the peak-to-peak intervals are equal to each other (S9), and the position information of the detection data is corrected using the calculated shift amount as the shift amount of the detection data to which the target peak belongs (S10)”.

PTL 2 describes “a method for determining a base sequence of a nucleic acid, the method including the following steps (A) to (C) in this order: (A) a basic peak extracting step of extracting basic peaks from electrophoretic data including respective peaks of four types of bases obtained by electrophoretically separating a sample nucleic acid; (B) a condition setting step of setting a search start basic peak at which a search starts and a peak-to-peak interval reference value in time-series data composed of the extracted basic peaks; and (C) a step of using the search start basic peak in the time-series data as a start point to sequentially scan an interval between adjacent basic peaks in forward and backward directions of the time-series and comparing the interval between the basic peaks to the peak-to-peak interval reference value and adding an interpolation peak to a peak missing section to determine the base sequence”.

### CITATION LIST

### Patent Literature

PTL 1: JP2002-228633A

PTL 2: WO2008/050426A

### Non-Patent Literature

NPL 1: Michael C. Giddings, Jessica Severin, Michael Westphall, Jiazhen Wu, and Lloyd M. Smith, “A Software System for Data Analysis in Automated DNA Sequencing”, Genome Res. 1998 June; 8(6):644-665

### SUMMARY OF INVENTION

### Technical Problem

Recently, a need for a reduction in a size of the electrophoresis device has increased. When the size of the electrophoresis device is reduced, an accuracy of a resolution of a signal decreases, and there is a problem in that it is difficult to correct the accuracy using a waveform of the signal.

When the accuracy of the resolution of a signal decreases, there is a case where the method of correcting mobility in the related art does not function appropriately. For example, in the case of a sample including a long mixed base sequence or in the case of a signal including a large noise, there is a case where the waveform of the signal is corrected to reduce overlap of waveforms, and an accurate base sequence cannot be identified.

An object of the invention is to provide a method for correcting mobility to solve the above-described problem.

### Solution to Problem

A representative example of the invention disclosed in the present application is as follows. That is, there is provided an analysis method of a base sequence that is executed by a gene analyzer for analyzing a base sequence of a sample using time-series data of signal intensities of a plurality of bases acquired by electrophoresing the sample, the gene analyzer managing an observation environment and mobility correction amount data for correcting a position in a time direction of the time-series data of the signal intensities of the plurality of bases in association with each other, and the analysis method of a base sequence including: a first step of allowing the gene analyzer to scale the mobility correction amount data associated with the observation environment different from a first observation environment to generate default mobility correction amount data when the gene analyzer receives time-series data of signal intensities of a plurality of bases acquired by electrophoresing the sample in the first observation environment; a second step of allowing the gene analyzer to correct the position in the time direction of the time-series data of the signal intensities of the plurality of bases using an optimization algorithm of a mobility correction amount and the default mobility correction amount data; and a third step of identifying the base sequence of the sample using the corrected time-series data of the signal intensities of the plurality of bases.

### Advantageous Effects of Invention

According to the invention, mobility correction with high analysis accuracy of a base sequence of a sample can be implemented. Objects, configurations, and effects other than those described above will be clarified by describing the following embodiments.

### BRIEF DESCRIPTION OF DRAWINGS

FIG. 1 is a diagram illustrating a configuration example of a gene analyzer according to a first embodiment.

FIG. 2 is a diagram illustrating a configuration example of an electrophoresis device according to the first embodiment.

FIG. 3 is a diagram illustrating an example of electrophoretic characteristic information stored in a storage device according to the first embodiment.

FIG. 4A is a diagram illustrating an example of mobility correction amount information stored in the storage device according to the first embodiment.

FIG. 4B is a diagram illustrating an example of the mobility correction amount information stored in the storage device according to the first embodiment.

FIG. 4C is a diagram illustrating an example of the mobility correction amount information stored in the storage device according to the first embodiment.

FIG. 5 is a flowchart illustrating the summary of a process that is executed by the gene analyzer according to the first embodiment.

FIG. 6 is a flowchart illustrating an electrophoresis process that is executed by the electrophoresis device according to the first embodiment.

FIG. 7 is a flowchart illustrating a mobility correction process that is executed by a data analyzer according to the first embodiment.

FIG. 8 is a diagram illustrating an image of a method of calculating a scale in the first embodiment.

FIG. 9 is a diagram illustrating an image of scaling of mobility correction amount data in the first embodiment.

FIG. 10A is a diagram illustrating an example of a method of modifying automatic mobility correction amount data according to the first embodiment.

FIG. 10B is a diagram illustrating an example of the method of modifying the automatic mobility correction amount data according to the first embodiment.

FIG. 11 is a diagram illustrating a configuration example of a gene analyzer according to a second embodiment.

FIG. 12 is a flowchart illustrating the summary of a data generation process that is executed by the gene analyzer according to the second embodiment.

FIG. 13 is a flowchart illustrating the summary of a mobility correction amount data generation process that is executed by the gene analyzer according to the second embodiment.

FIG. 14 is a diagram illustrating an image of electrophoretic characteristic data and mobility correction amount data stored in a storage device according to a third embodiment.

### DESCRIPTION OF EMBODIMENTS

Hereinafter, embodiments of the invention will be described using the drawings. Note that the contents described in the following embodiments are not intended to limit the invention. A person skilled in the art can easily understand that a specific configuration of the invention can be changed within a range not departing from the spirit of the invention.

In a configuration of the invention that is described below, the same or similar components or functions will be represented by the same reference numerals, and the description thereof will not be repeated.

In the present specification and the like, the expression “first”, “second”, “third”, or the like is added to distinguish between components, and does not always limit the number or order thereof.

For easy understanding of the invention, the position, size, shape, range, and the like of each of the components illustrated in the drawings do not necessarily represent the actual ones. Accordingly, the invention is not necessarily limited to the position, size, shape, range, and the like illustrated in the drawings.

### First Embodiment

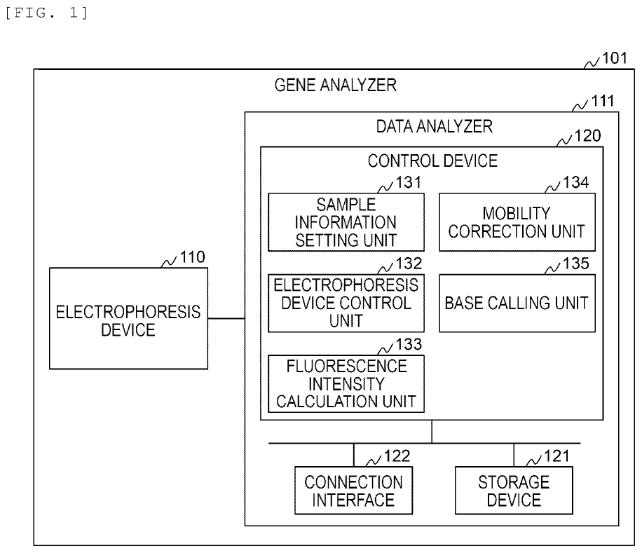

FIG. 1 is a diagram illustrating a configuration example of a gene analyzer 100 according to a first embodiment.

The gene analyzer 100 includes an electrophoresis device 110 and a data analyzer 111. The electrophoresis device 110 and the data analyzer 111 are communicably connected using a communication cable.

The data analyzer 111 includes a control device 120, a storage device 121, and a connection interface 122.

The control device 120 executes control and data processing of the electrophoresis device 110. The control device 120 is, for example, a central processing unit (CPU) and a graphics processing unit (GPU).

The storage device 121 stores a program to be executed by the control device 120, setting information of the electrophoresis device 110, information used for various processes, and the like. The storage device 121 is, for example, a memory.

The connection interface 122 is an interface that connects an input device and an output device, or an interface that is connected to an external device via a network. The data analyzer 111 presents information to a user or receives information input from the user via the connection interface 122.

By executing the program stored in the storage device 121, the control device 120 operates as a sample information setting unit 131, an electrophoresis device control unit 132, a fluorescence intensity calculation unit 133, a mobility correction unit 134, and a base calling unit 135. In the following description, when a functional unit is used as a subject to describe a process, it can be considered that the control device 120 executes the program.

The electrophoresis device 110 electrophoreses a sample (DNA fragment) to acquire electrophoretic data. The electrophoretic data is time-series data of a brightness value of the DNA fragment labeled with a fluorescent dye.

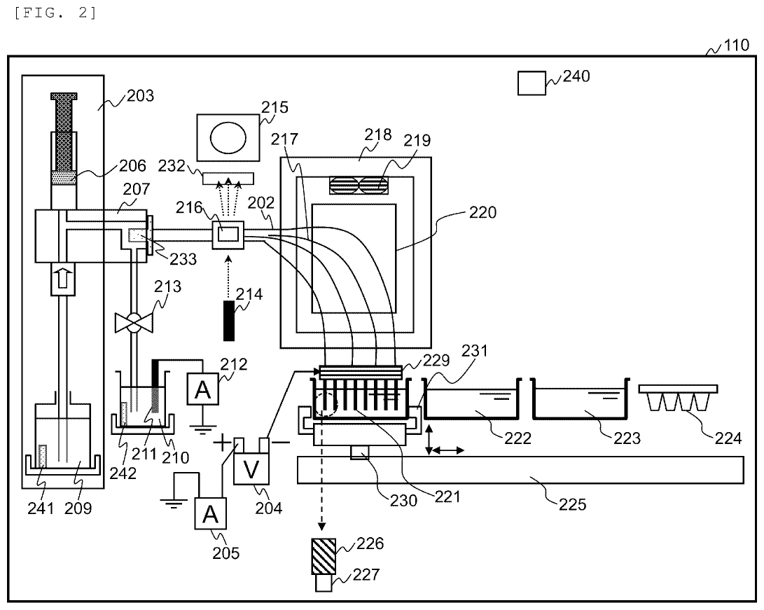

Here, a configuration of the electrophoresis device 110 will be described. FIG. 2 is a diagram illustrating a configuration example of the electrophoresis device 110 according to the first embodiment.

The electrophoresis device 110 includes a detection unit 216, a thermostat 218, a transport device 225, a high voltage power supply 204, a first ammeter 205, an anode-side electrode 211, a second ammeter 212, a capillary array 217, and a pump mechanism 203.

The capillary array 217 is a replacement member including a plurality of (for example, eight) capillaries 202, and includes a load header 229, the detection unit 216, and a capillary head 233. Along with breakage or deterioration of the quality of the capillaries 202, the capillary array 217 can be replaced with a new one.

The capillary 202 is configured by a glass tube having an inner diameter of several tens to several hundreds of microns and having an outer diameter of several hundreds of microns, and a surface thereof is coated with polyimide to improve the strength. Note that a light irradiation unit that emits a laser beam has a structure where the polyimide coating is removed such that the emitted light easily leaks from the inside to the outside. The capillary 202 is filled with a separation medium for applying a difference in migration speed during electrophoresis. As the separation medium, both of a flowable medium and a non-flowable medium can be used. However, in the first embodiment, a flowable polymer is used.

The high voltage power supply 204 applies a high voltage to the capillary 202. The first ammeter 205 detects a current generated from the high voltage power supply 204. The second ammeter 212 detects a current flowing through the anode-side electrode 211.

An optical detection unit that detects information light acquired from the sample is configured by a light source 214 that emits excitation light to the detection unit 216, an optical detection unit 215 for detecting emission in the detection unit 216, and a diffraction grating 232. The detection unit 216 is a member that acquires information depending on the sample.

When the sample in the capillary 202 separated by electrophoresis is detected, by emitting the excitation light from the light source 214 to the detection unit 216, fluorescence having a wavelength depending on the sample is likely to be formed as the information light. The diffraction grating 232 disperses the information light in a wavelength direction, and the optical detection unit 215 detects the dispersed information light to analyze the sample.

Each capillary cathode end 227 is fixed through a metallic hollow electrode 226, and a tip of the capillary 202 protrudes from the hollow electrode 226 by about 0.5 mm. All of the hollow electrodes 226 in the capillaries 202 are integrated and mounted on the load header 229. All of the hollow electrodes 226 are electrically connected to the high voltage power supply 204 mounted on the device main body and, when it is necessary to apply a voltage, for example, during electrophoresis or sample introduction, functions as a cathode electrode.

Capillary end portions (other end portion) opposite to the capillary cathode ends 227 are bundled into one by the capillary head 233. The capillary head 233 is connected to a block 207 with pressure-resistance and airtightness. A high voltage generated by the high voltage power supply 204 is applied between the load header 229 and the capillary head 233. The capillaries 202 are filled with a new polymer from the other end portions by a syringe 206. The refill of the polymer in the capillaries 202 is executed per measurement to improve the performance of the measurement.

The pump mechanism 203 is configured by the syringe 206 and a mechanical system for pressurizing the syringe 206 and injects the polymer into the capillaries 202.

The block 207 is a connection portion for connecting the syringe 206, the capillary array 217, an anode buffer container 210, and a polymer container 209 to communicate with each other.

To keep the capillaries 202 in the thermostat 218 at a constant temperature, the thermostat 218 is covered with a heat insulating material, and the temperature is controlled by a heating/cooling mechanism 220. A fan 219 circulates and stirs air in the thermostat 218, and the temperature of the capillary array 217 is kept positionally uniform and constant.

The transport device 225 transports various containers to the capillary cathode ends 227. The transport device 225 includes three electric motors and a linear actuator and is movable in three axis directions including vertical, horizontal, and depth directions. At least one container can be placed on a moving stage 230 of the transport device 225. The moving stage 230 includes an electric grip 231 and can grip and release each of the containers. Therefore, a buffer container 221, a cleaning container 222, a waste solution container 223, and a sample plate 224 can be transported to the capillary cathode ends 227 as necessary. Unnecessary containers are stored in a predetermined storage in the electrophoresis device 110.

Using the data analyzer 111, the user can control various functions of the electrophoresis device 110 to acquire the electrophoretic data detected by the optical detection unit.

In the electrophoresis device 110, a sensor for acquiring information regarding an observation environment that affects electrophoresis (observation environment information) may be present. The electrophoresis device 110 in FIG. 2 includes an internal sensor 240, a polymer sensor 241, and a buffer solution sensor 242.

The internal sensor 240 is a sensor for acquiring information regarding an internal environment of the electrophoresis device 110 and, for example, is a temperature sensor, a humidity sensor, an air pressure sensor, and the like.

The polymer sensor 241 is a sensor for acquiring information regarding the quality of the polymer and is, for example, a PH sensor or an electrical conductivity sensor. The polymer sensor 241 is installed in the polymer container 209 in FIG. 2, but the installation position is not limited thereto.

The buffer solution sensor 242 is a sensor for acquiring information regarding the quality of the buffer solution and is, for example, a temperature sensor. The buffer solution sensor 242 is installed in the anode buffer container 210 in FIG. 2, but the installation position is not limited thereto. For example, the buffer solution sensor 242 may be set in the buffer container 221.

FIG. 3 is a diagram illustrating an example of electrophoretic characteristic information stored in the storage device 121 according to the first embodiment.

For each base type, the storage device 121 stores electrophoretic characteristic information that manages electrophoretic characteristic data representing a relationship between a migration time (t) and a base position (p). The electrophoretic characteristic information stores electrophoretic characteristic data for each observation environment in the electrophoresis device 110.

The electrophoretic characteristic data is managed as a function Y(p). In the present embodiment, a parameter representing the function is managed instead of the function itself. FIG. 3 illustrates electrophoretic characteristic data where voltages to be applied from the high voltage power supply 204 are different. Y₅(p) represents electrophoretic characteristic data where the voltage is 5.0 kV, Y₈(p) represents electrophoretic characteristic data where the voltage is 8.0 kV, and Y₁₁(p) represents electrophoretic characteristic data where the voltage is 11.0 kV.

The electrophoretic characteristic information may be stored in the storage device 121 in advance, or may be generated using electrophoretic data obtained by electrophoresing a sample having a known length such as a size standard using the electrophoresis device 110.

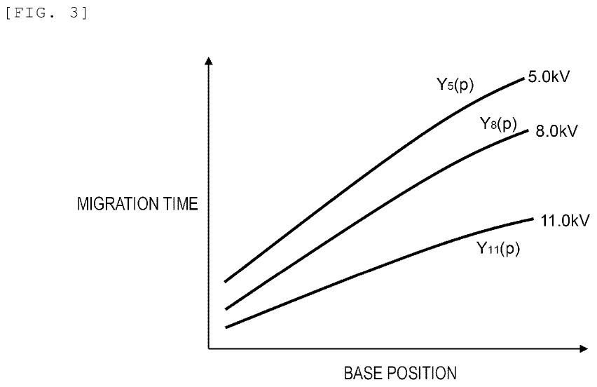

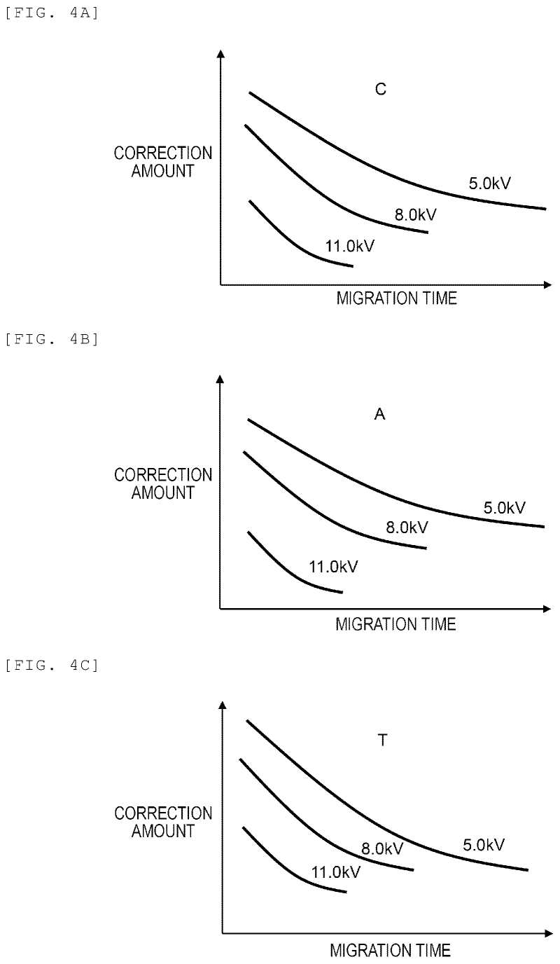

FIGS. 4A, 4B, and 4C are diagrams illustrating examples of mobility correction amount information stored in the storage device 121 according to the first embodiment.

For each base type, the storage device 121 stores mobility correction amount information that manages mobility correction amount data representing a relationship between a migration time and a mobility correction amount. The mobility correction amount information stores default mobility correction amount data for each observation environment in the electrophoresis device 110. In the first embodiment, the mobilities of a C (cytosine) base, an A (adenine) base, and a T (thymine) base are corrected with respect to a G (guanine) base. Accordingly, the storage device 121 stores mobility correction amount information of each of the C base, the A base, and the T base.

FIG. 4A illustrates the mobility correction amount information of the C base, FIG. 4B illustrates the mobility correction amount information of the A base, and FIG. 4C illustrates the mobility correction amount information of the T base. A curve represents the default mobility correction amount data.

The mobility correction amount information may be stored in the storage device 121 in advance, or may be generated using electrophoretic data obtained by electrophoresing a sample having a known length such as a size standard using the electrophoresis device 110.

FIG. 5 is a flowchart illustrating the summary of a process that is executed by the gene analyzer 100 according to the first embodiment.

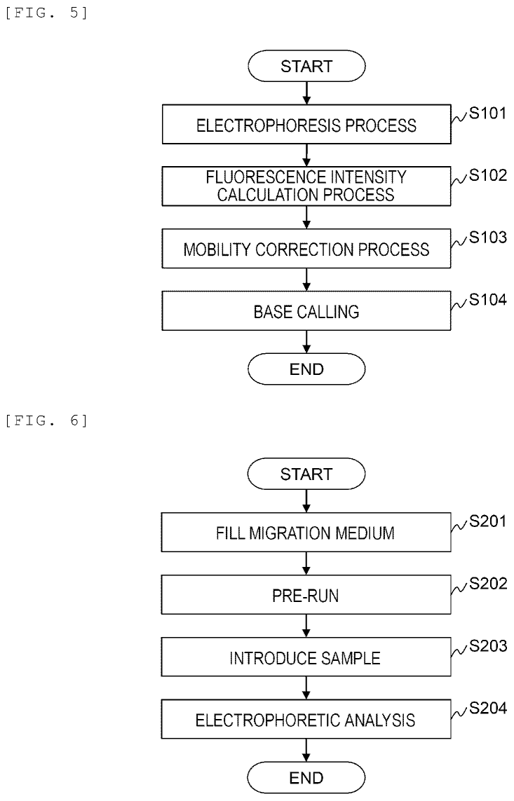

The electrophoresis device 110 of the gene analyzer 100 executes an electrophoresis process on a sample to be analyzed (Step S101). The details of the electrophoresis process will be described using FIG. 6.

Next, the data analyzer 111 of the gene analyzer 100 executes a fluorescence intensity calculation process using the electrophoretic data (Step S102). Specifically, the fluorescence intensity calculation unit 133 calculates time-series data of a fluorescence intensity of a fluorescent dye from the electrophoretic data, and detects a center position, a height, a width, and the like of a peak from the time-series data of the fluorescence intensity.

Next, the data analyzer 111 of the gene analyzer 100 executes a mobility correction process on the time-series data of the fluorescence intensity (Step S103). The details of the mobility correction process will be described using FIGS. 7 to 10. The gene analyzer 100 according to the first embodiment has a characteristic in the mobility correction process as described below.

Next, the data analyzer 111 of the gene analyzer 100 executes base calling using the corrected time-series data of the fluorescence intensity based on the result of the mobility correction process (Step S104). Specifically, the base calling unit 135 identifies a base sequence of the sample using the corrected time-series data of the fluorescence intensity.

FIG. 6 is a flowchart illustrating the electrophoresis process that is executed by the electrophoresis device 110 according to the first embodiment.

The user sets a sample to be analyzed and a reagent to the electrophoresis device 110 and instructs the start of the electrophoresis process via the connection interface 122. The setting of the sample is executed in the following procedure.

The user fills the buffer container 221 and the anode buffer container 210 with a buffer solution for forming a part of a conduction path. The buffer solution is, for example, an electrolytic solution that is commercially available from each manufacturer for electrophoresis. The user dispenses the sample to be analyzed into a well of the sample plate 224. The sample is, for example, a PCR product of DNA. The user dispenses a cleaning solution for cleaning the capillary cathode ends 227 into the cleaning container 222. The cleaning solution is, for example, pure water. The user injects a migration medium for electrophoresing the sample into the syringe 206. The migration medium is, for example, a polyacrylamide separation gel or a polymer that is commercially available from each manufacturer for electrophoresis. When deterioration of the capillaries 202 is expected or when the lengths of the capillaries 202 are changed, the user replaces the capillary array 217.

Here, as the samples to be set to the sample plate 224, for example, in addition to the actual sample of DNA to be analyzed, a positive control, a negative control, and an allelic ladder can be set, and the samples are electrophoresed in the different capillaries 202.

The positive control is, for example, a PCR product including known DNA and is a sample for a control experiment verifying that DNA is correctly amplified by PCR. The negative control is a PCR product not including DNA and is a sample for a control experiment for verifying that a contamination such as DNA of the user and dust is not generated in an amplification product of PCR. The allelic ladder is an artificial sample including a large number of bases that may be included in a DNA marker, and is generally provided from a reagent manufacturer as a reagent kit for a DNA test. The allelic ladder is used to finely adjust a correspondence between DNA fragment lengths of individual DNA markers and alleles.

A known DNA fragment called a size standard that is labeled with a specific fluorescent dye is mixed with all of the actual sample, the positive control, the negative control, and the allelic ladder. The type of the fluorescent dye allotted to the size standard varies depending on the reagent kit to be used.

The user designates the type of the allelic ladder, the type of the size standard, the type of the fluorescent reagent, the type of sample set to the well on the sample plate 224 corresponding to each of the capillaries 202, and the like. In the first embodiment, the type of any one of the actual sample, the positive control, the negative control, and the allelic ladder is designated. The setting of the information is input to the sample information setting unit 131 via the connection interface 122 of the data analyzer 111.

Hereinabove, the setting of the sample has been described.

The electrophoresis device control unit 132 transmits a signal for instructing the start of the analysis to the electrophoresis device 110. When the signal is received, the electrophoresis device 110 starts the electrophoresis process described below.

The electrophoresis device 110 fills the capillary 202 with a new migration medium to form a migration path (Step S201). The filling of the migration medium may be automatically executed after the start of the analysis or may be sequentially executed based on a control signal transmitted from the electrophoresis device control unit 132.

Specifically, the electrophoresis device 110 allows the transport device 225 to transport the waste solution container 223 to a position immediately below the load header 229, closes a solenoid valve 213, and receives the used migration medium discharged from the capillary cathode end 227. The electrophoresis device 110 drives the syringe 206 to fill the capillary 202 with the new migration medium, and discards the used migration medium. Finally, the electrophoresis device 110 immerses the capillary cathode end 227 in the cleaning solution in the cleaning container 222 to clean the capillary cathode end 227 contaminated with the migration medium.

Next, the electrophoresis device 110 applies a predetermined voltage to the migration medium, and executes pre-run for adjusting the migration medium to a state suitable for electrophoresis (Step S202). The pre-run may be automatically executed or may be sequentially executed based on a control signal transmitted from the electrophoresis device control unit 132.

Specifically, the electrophoresis device 110 allows the transport device 225 to immerse the capillary cathode end 227 in the buffer solution in the buffer container 221 to form a conduction path. The electrophoresis device 110 allows the high voltage power supply 204 to apply a voltage of several to several tens of kilovolts to the migration medium for several to several tens of minutes to adjust the migration medium to a state suitable for electrophoresis. Finally, the electrophoresis device 110 immerses the capillary cathode end 227 in the cleaning solution in the cleaning container 222 to clean the capillary cathode end 227 contaminated with the buffer solution.

Next, the electrophoresis device 110 introduces the sample (Step S203). The introduction of the sample may be automatically executed or may be sequentially executed based on a control signal transmitted from the electrophoresis device control unit 132.

Specifically, the electrophoresis device 110 allows the transport device 225 to immerse the capillary cathode end 227 in the sample held in the well of the sample plate 224 and subsequently opens the solenoid valve 213. As a result, a conduction path is formed, and a state where the sample components can be introduced into the migration path is established. The electrophoresis device 110 allows the high voltage power supply 204 to apply a pulse voltage to the conduction path to introduce the sample components into the migration path. Finally, the electrophoresis device 110 immerses the capillary cathode end 227 in the cleaning solution in the cleaning container 222 to clean the capillary cathode end 227 contaminated with the sample.

Next, the electrophoresis device 110 executes electrophoretic analysis of separating and analyzing the sample components in the sample (Step S204). The electrophoretic analysis may be automatically executed or may be sequentially executed based on a control signal transmitted from the electrophoresis device control unit 132.

Specifically, the electrophoresis device 110 allows the transport device 225 to immerse the capillary cathode end 227 in the buffer solution in the buffer container 221 to form a conduction path. The electrophoresis device 110 allows the high voltage power supply 204 to apply a high voltage of about 15 kV to the conduction path to generate an electric field in the migration path. Due to the generated electric field, each of the sample components in the migration path moves to the detection unit 216 at a speed depending on the characteristics of each of the sample components. That is, the sample components are separated by a difference in moving speed. The sample components arrived at the detection unit 216 are detected in order of arrival. For example, when the sample includes a large number of DNA fragments having different numbers of bases, a difference in moving speed is generated depending on the number of bases, and the DNA fragments arrive at the detection unit 216 in order from the DNA fragment having the smallest number of bases. The fluorescent dye depending on a terminal base sequence is attached to each of the DNA fragments. By emitting the excitation light from the light source 214 to the detection unit 216, fluorescence having a wavelength depending on the sample is formed and emitted to the outside. The electrophoresis device 110 allows the optical detection unit 215 to detect the fluorescence. During the electrophoretic analysis, the optical detection unit 215 detects the fluorescence at a constant time interval and transmits the image data to the data analyzer 111. To reduce the amount of information to be transmitted, not only the image data but also a brightness of only a partial region in the image data may be transmitted. For example, for each of the capillaries 202, a brightness value that is sampled from only a wavelength position at a constant interval may be transmitted. The data transmitted from the electrophoresis device 110 is time-series data of brightness values of each of the capillaries 202 and is stored in the storage device 121.

When predetermined image data is acquired, the electrophoresis device 110 stops the application of the voltage and ends the electrophoretic analysis. Hereinabove, the electrophoresis process has been described.

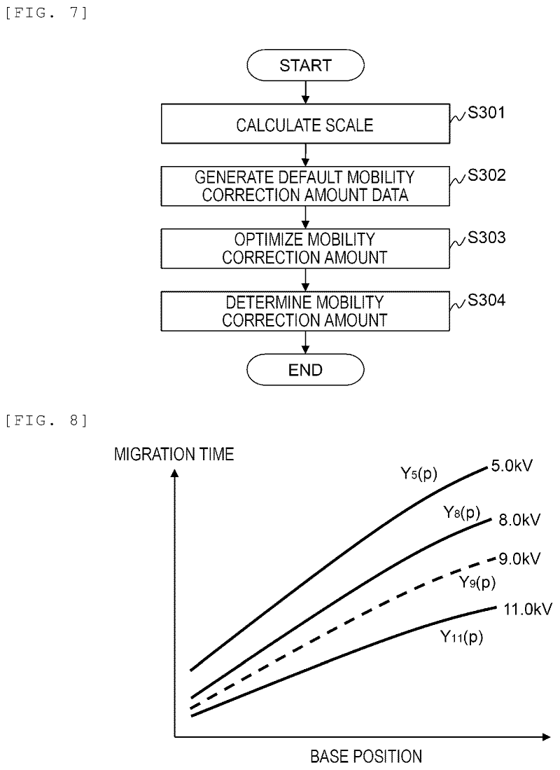

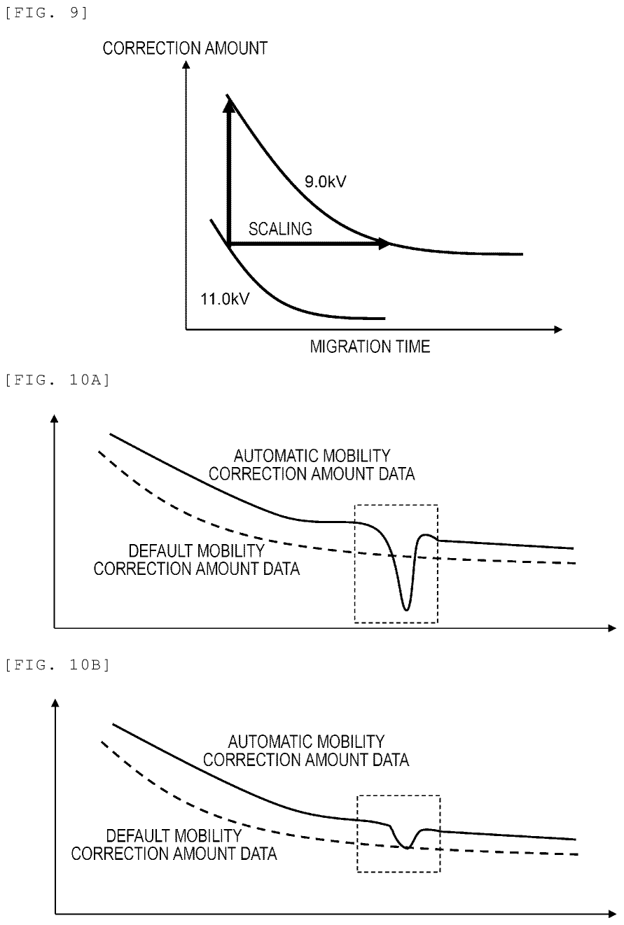

FIG. 7 is a flowchart illustrating the mobility correction process that is executed by the data analyzer 111 according to the first embodiment. FIG. 8 is a diagram illustrating an image of a method of calculating a scale in the first embodiment. FIG. 9 is a diagram illustrating an image of scaling of mobility correction amount data in the first embodiment. FIGS. 10A and 10B are diagrams illustrating examples of a method of modifying automatic mobility correction amount data according to the first embodiment.

The mobility correction unit 134 calculates a scale based on the observation environment information of the electrophoresis device 110 (Step S301). Here, the method of calculating a scale focusing on a voltage in the observation environment will be described.

(S301-1) The mobility correction unit 134 refers to the electrophoretic characteristic information to search for electrophoretic characteristic data associated with a voltage (target voltage) in the observation environment information. When the electrophoretic characteristic data associated with the target voltage is present, the default mobility correction amount data associated with the target voltage is present. Therefore, the mobility correction unit 134 ends the process of Step S301. Here, the calculation of the scale is not executed.

(S301-2) When the electrophoretic characteristic data associated with the target voltage is not present, the mobility correction unit 134 generates electrophoretic characteristic data of the target voltage (target electrophoretic characteristic data) using electrophoretic characteristic data of a voltage having a small difference from the target voltage. As such, electrophoretic characteristic data of an actual observation environment is estimated using electrophoretic characteristic data of an observation environment similar to the actual observation environment. Here, being similar to the observation environment represents being similar to a combination of physical quantities representing the observation environment.

For example, when the target voltage is 9.0 kV, as illustrated in FIG. 8, target electrophoretic characteristic data is generated using electrophoretic characteristic data Y₈(p) of 8.0 kV and electrophoretic characteristic data Y₁₁(p) of 11.0 kV. When linear interpolation is used, the electrophoretic characteristic data of the target voltage is generated from Expression (1), for example.

$$\begin{matrix}[ Numeral 1 ] & \\ {Y}_{9} ( p ) = \frac{2}{3} {Y}_{8} ( p ) + \frac{1}{3} {Y}_{11} ( p ) & ( 1 )\end{matrix}$$

(S301-3) The mobility correction unit 134 calculates a scale between the electrophoretic characteristic data used to generate the target electrophoretic characteristic data at time t and the target electrophoretic characteristic data.

For example, when the target voltage is 9.0 kV, a scale S₈(t) for the electrophoretic characteristic data Y₈(p) of 8.0 kV at the time t is provided from Expression (2), and a scale S₁₁(t) for the electrophoretic characteristic data Y₁₁(p) of 11.0 kV at the time t is provided from Expression (3).

$$\begin{matrix}[ Numeral 2 ] & \\ {S}_{8} ( t ) = \frac{{Y}_{9} ( p )}{{Y}_{8} ( p )} & ( 2 )\end{matrix}$$

$$\begin{matrix}[ Numeral 3 ] & \\ {S}_{11} ( t ) = \frac{{Y}_{9} ( p )}{{Y}_{11} ( p )} & ( 3 )\end{matrix}$$

Hereinabove, the method of calculating the scale has been described.

Next, the mobility correction unit 134 generates the default mobility correction amount data (Step S302). Here, the method of calculating default mobility correction amount data focusing on a voltage in the observation environment will be described.

(S302-1) The mobility correction unit 134 refers to the mobility correction amount information of each base to search for default mobility correction amount data associated with a voltage (target voltage) in the observation environment information. When the default mobility correction amount data associated with the target voltage is present, the mobility correction unit 134 ends the process of Step S302. Here, the searched default mobility correction amount data is used as it is.

(S302-2) When the default mobility correction amount data associated with the target voltage is not present, in Step S301, the mobility correction unit 134 identifies a voltage of electrophoretic characteristic data used for generating the target electrophoretic characteristic data. The mobility correction unit 134 refers to the mobility correction amount information of each base to acquire default mobility correction amount data associated with the identified voltage.

(S302-3) As illustrated in FIG. 9, the mobility correction unit 134 multiplies the scale associated with the identified voltage by each point of the default mobility correction amount data to calculate the default mobility correction amount data of the target voltage.

For example, when the target voltage is 9.0 kV, the mobility correction unit 134 multiplies the default mobility correction amount data of 8.0 kV by the scale S₈(t) to calculate first default mobility correction amount data, and multiplies the default mobility correction amount data of 11.0 kV by the scale S₁₁(t) to calculate second default mobility correction amount data. The mobility correction unit 134 calculates the average of the first default mobility correction amount data and the second default mobility correction amount data as default mobility correction amount data of 9.0 kV.

As such, in the default mobility correction amount data of each observation environment in the first embodiment, the scale relationship is present.

Hereinabove, the method of calculating the default mobility correction amount data has been described.

Next, the mobility correction unit 134 executes optimization of the mobility correction amount on the time-series data of the fluorescence intensity (Step S303).

For the optimization of the mobility correction amount, a well-known optimization algorithm of the mobility correction amount may be used. Therefore, although the detailed description is not made, for example, the optimization algorithm is considered to be as follows.

The mobility correction unit 134 sets a block having a predetermined time size and, while moving the block in a time direction for the time-series data of the fluorescence intensity, searches for the mobility correction amount of each base such that overlap of waveforms of bases in the block is minimized. The result of the search process is plotted, and interpolation is smoothly executed between the plotted points to acquire the mobility correction amount data (automatic mobility correction amount data).

The default mobility correction amount data may be used as an initial value of the optimization.

Next, the mobility correction unit 134 determines the mobility correction amount data based on the automatic mobility correction amount data and the default mobility correction amount data (Step S304). For example, the process is considered to be as follows.

(Process 1) When a difference between the automatic mobility correction amount data and the default mobility correction amount data is large, the mobility correction unit 134 adopts the default mobility correction amount data, and when the difference is small, the mobility correction unit 134 adopts the automatic mobility correction amount data. The above-described difference can be evaluated based on a maximum value of the difference between the automatic mobility correction amount data and the default mobility correction amount data, a total value of the differences at each point between the automatic mobility correction amount data and the default mobility correction amount data, and the like. In a portion where the difference between the automatic mobility correction amount data and the default mobility correction amount data is small, the automatic mobility correction amount data may be adopted. In a portion where the difference between the automatic mobility correction amount data and the default mobility correction amount data is large, the default mobility correction amount data may be adopted.

(Process 2) As illustrated in FIG. 10A, the mobility correction unit 134 identifies a portion (a rectangular region of a dotted line) where the difference between the automatic mobility correction amount data (a graph of a solid line) and the default mobility correction amount data (a graph of a dotted line) is large and a variation is large. As illustrated in FIG. 10B, the mobility correction unit 134 corrects the identified portion such that the difference from the default mobility correction amount data is small and the variation is small.

In the automatic mobility correction process, the mobility correction amount is calculated such that the overlap is reduced. Therefore, when the mixed base sequence is long, there may be a case where an appropriate mobility correction is not calculated. Here, the difference from the default mobility correction amount data is likely to be large.

The mobility correction unit 134 according to the first embodiment executes the automatic correction using the default mobility correction amount data as a limit of the correction. As a result, even for a sample including a long mixed base sequence or electrophoretic data including a large noise, the mobility can be accurately corrected.

The mobility correction amount data that is actually used is adjusted based on the default mobility correction amount data, and thus has a characteristic in which a part or the entirety is similar to the default mobility correction amount data.

In the first embodiment, default mobility correction amount data in one observation environment is generated by scaling default mobility correction amount data in different observation environments. As a result, various observation environments can be handled.

### Second Embodiment

In a second embodiment, the data analyzer 111 generates electrophoretic characteristic data and default mobility correction amount data for each observation environment. Hereinafter, a difference of the second embodiment from the first embodiment will be mainly described.

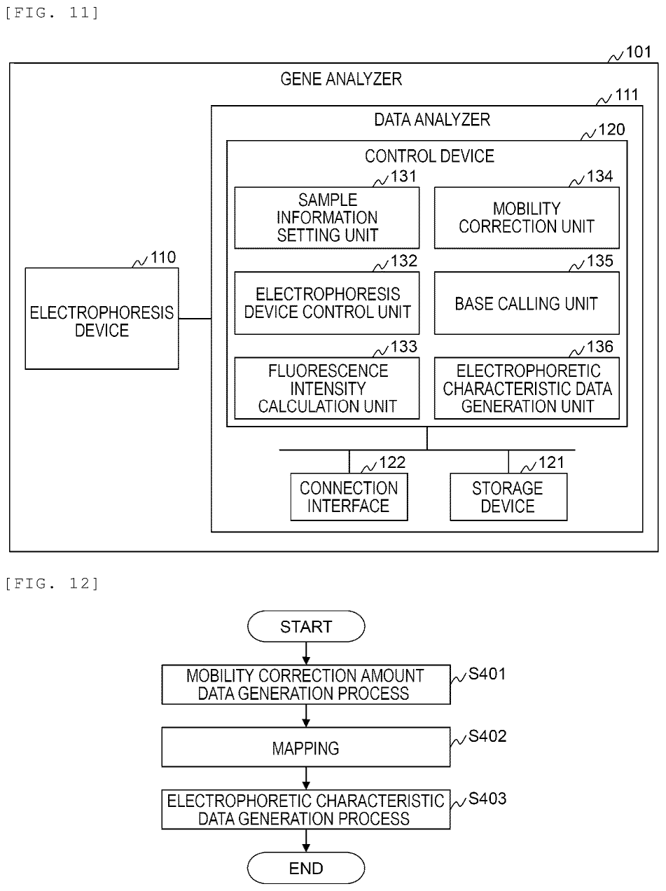

FIG. 11 is a diagram illustrating a configuration example of the gene analyzer 100 according to the second embodiment.

The configuration of the electrophoresis device 110 according to the second embodiment is the same as that of the first embodiment. A hardware configuration of the data analyzer 111 according to the second embodiment is the same as the first embodiment.

A software configuration of the data analyzer 111 according to the second embodiment is partially different from that of the first embodiment. Specifically, the data analyzer 111 according to the second embodiment is different from that of the first embodiment in that the data analyzer 111 according to the second embodiment includes an electrophoretic characteristic data generation unit 136. The electrophoretic characteristic data generation unit 136 generates electrophoretic characteristic data and default mobility correction amount data.

The process that is executed by the gene analyzer 100 according to the second embodiment is the same as that of the first embodiment. The electrophoresis process that is executed by the electrophoresis device 110 according to the second embodiment is the same as that of the first embodiment. The mobility correction process that is executed by the data analyzer 111 according to the second embodiment is the same as that of the first embodiment.

The storage device 121 stores electrophoretic characteristic data, mobility correction amount data, and a base sequence for reference of any sample in any observation environment. The sample is preferably a sample having a single-base sequence where base calling is easy.

FIG. 12 is a flowchart illustrating the summary of the data generation process that is executed by the gene analyzer 100 according to the second embodiment.

When an instruction is received from the user, the gene analyzer 100 executes the data generation process.

The gene analyzer 100 executes the mobility correction amount data generation process (Step S401). In the mobility correction amount data generation process, mobility correction amount data is generated for a plurality of observation environments, and base calling of a base sequence is executed. The details of the process will be described using FIG. 12.

Next, the gene analyzer 100 executes mapping between a reference base sequence and a base sequence in any observation environment (Step S402).

Specifically, the electrophoretic characteristic data generation unit 136 of the data analyzer 111 calculates a base position of a G base in the base sequence in each observation environment by the mapping.

Next, the gene analyzer 100 executes the electrophoretic characteristic data generation process (Step S403). Specifically, the following process is executed.

(S403-1) The electrophoretic characteristic data generation unit 136 of the data analyzer 111 plots points in a space where the axes represent a base position and a migration time based on a base sequence of one observation environment.

(S403-2) The electrophoretic characteristic data generation unit 136 may execute interpolation between the points plotted in S403-1 as necessary.

(S403-3) The electrophoretic characteristic data generation unit 136 acquires an approximate expression from the plotted points. The electrophoretic characteristic data generation unit 136 stores parameters or the like of the approximate expression in the storage device 121 as the electrophoretic characteristic data in association with the observation environment.

Hereinabove, the process of Step S403 has been described.

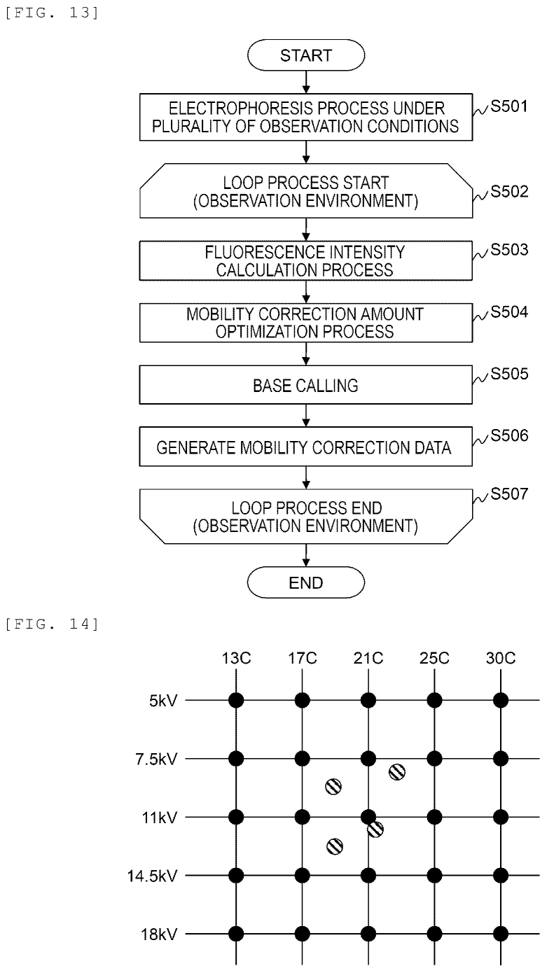

FIG. 13 is a flowchart illustrating the summary of the mobility correction amount data generation process that is executed by the gene analyzer 100 according to the second embodiment.

The electrophoresis device 110 of the gene analyzer 100 executes the electrophoresis process of the sample for a plurality of observation environments (Step S501). The electrophoresis process of the second embodiment is the same as that of the first embodiment.

The observation environment is designated by the user. By executing the electrophoresis process multiple times for one observation environment, a plurality of pieces of electrophoretic data are acquired.

Next, the data analyzer 111 of the gene analyzer 100 starts a loop process of the observation environment (Step S502). Specifically, the data analyzer 111 selects one observation environment.

Next, the data analyzer 111 of the gene analyzer 100 executes a fluorescence intensity calculation process using the plurality of pieces of electrophoretic data (Step S503). The fluorescence intensity calculation process of the second embodiment is the same as that of the first embodiment.

Next, the data analyzer 111 of the gene analyzer 100 executes optimization of the mobility correction amount for the time-series data of each of the fluorescence intensities (Step S504). For the optimization of the mobility correction amount in the second embodiment, a well-known technique is used.

Next, the data analyzer 111 of the gene analyzer 100 executes base calling using the corrected time-series data of the fluorescence intensity (Step S505).

Next, the data analyzer 111 of the gene analyzer 100 generates the mobility correction amount data of the observation environment (Step S506).

Specifically, the electrophoretic characteristic data generation unit 136 calculates an average of the plurality of pieces of mobility correction amount data calculated in Step S504. The electrophoretic characteristic data generation unit 136 stores the calculated mobility correction amount data in the storage device 121 in association with the observation environment. The mobility correction amount data where error is determined in the base calling of the base sequence is excluded.

Next, the data analyzer 111 of the gene analyzer 100 determines whether the process is completed for all of the observation environments (Step S507).

Next, when the process is not completed for all of the observation environments, the data analyzer 111 of the gene analyzer 100 returns to Step S502 and executes the same process. When the process is completed for all of the observation environments, the gene analyzer 100 ends the mobility correction amount data generation process.

According to the second embodiment, by preparing the electrophoretic characteristic data of the plurality of observation environments, the cost for the calculation of the scale and the calculation of the mobility correction amount data can be reduced.

### Third Embodiment

In the third embodiment, the data analyzer 111 adds the electrophoretic characteristic data and the mobility correction amount calculated in the actual analysis. Hereinafter, a difference of the third embodiment from the first embodiment will be mainly described.

The configuration of the gene analyzer 100 according to the third embodiment is the same as that of the first embodiment. The configurations of the electrophoresis device 110 and the data analyzer 111 according to the third embodiment are the same as those of the first embodiment.

In the third embodiment, the process that is executed by the gene analyzer 100 is partially different. Specifically, after executing the base calling (Step S104), the data analyzer 111 stores the electrophoretic characteristic data and the mobility correction amount data calculated in the series of processes in the storage device 121 in association with the observation environment based on an instruction of the user.

FIG. 14 is a diagram illustrating an image of the electrophoretic characteristic data and the mobility correction amount data stored in the storage device 121 according to the third embodiment.

FIG. 14 illustrates a distribution of data in a space representing an observation environment configured by a temperature and a voltage. The initial storage device 121 stores electrophoretic characteristic data and mobility correction amount data of observation environments associated with black circles. It can be seen that electrophoretic characteristic data and mobility correction amount data of observation environments associated with circles of oblique lines are added in response to an instruction of the user.

The electrophoresis process and the mobility correction process of the third embodiment are the same as those of the first embodiment. The gene analyzer 100 according to the third embodiment may execute the data generation process.

According to the third embodiment, by adding the electrophoretic characteristic data and the mobility correction amount data depending on a change over time and an actual use environment of the gene analyzer 100, the accuracy and the cost of base calling can be reduced.

The invention is not limited to the embodiment and includes various modification examples. For example, in the above embodiments, the configurations have been described in detail to easily describe the invention, and the invention does not necessarily include all the configurations described above. Addition, deletion, and replacement of another configuration can be made for a part of the configuration each of the embodiments.

Some or all of the above-described respective configurations, functions, processing units, processing means, and the like may be realized by hardware, for example, by designing an integrated circuit. The invention can also be implemented by program codes of software that implements the functions of the embodiments. Here, a storage medium that stores the program codes is provided to a computer, and a processor in the computer reads the program code stored in the storage medium. Here, the program code itself read from the storage medium implements the functions of the embodiments, and the program code itself and the storage medium recording the program code configure the invention. As the storage medium for supplying the program code, for example, a flexible disk, a CD-ROM, a DVD-ROM, a hard disk, a solid state drive (SSD), an optical disk, a magneto-optical disk, a CD-R, a magnetic tape, a non-volatile memory card, or a ROM is used.

The program codes implementing the functions described in the embodiments can be implemented in a wide range of programs or script languages such as assembler, C/C++, perl, Shell, PHP, Python, and Java.

The program codes of the software implementing the functions of the embodiments may be distributed via a network such that the program codes are stored in storage means such as a hard disk or a memory of a computer or in a storage medium such as a CD-RW or a CD-R, and a processor in the computer may read and execute the program codes stored in the storage means or the storage medium.

In the embodiments, the drawings illustrate control lines and information lines as considered necessary for explanations but do not illustrate all control lines or information lines in the products. All the configurations may be interconnected.

1. An analysis method of a base sequence that is executed by a gene analyzer for analyzing a base sequence of a sample using time-series data of signal intensities of a plurality of bases acquired by electrophoresing the sample,
the gene analyzer managing an observation environment and mobility correction amount data for correcting a position in a time direction of the time-series data of the signal intensities of the plurality of bases in association with each other, and
the analysis method of a base sequence comprising:
a first step of allowing the gene analyzer to scale the mobility correction amount data associated with the observation environment different from a first observation environment to generate default mobility correction amount data when the gene analyzer receives time-series data of signal intensities of a plurality of bases acquired by electrophoresing the sample in the first observation environment;
a second step of allowing the gene analyzer to correct the position in the time direction of the time-series data of the signal intensities of the plurality of bases using an optimization algorithm of a mobility correction amount and the default mobility correction amount data; and
a third step of identifying the base sequence of the sample using the corrected time-series data of the signal intensities of the plurality of bases.

2. The analysis method of a base sequence according to claim 1, wherein
the gene analyzer manages an observation environment and electrophoretic characteristic data representing a relationship between a position of the base depending on electrophoresis and a migration time in association with each other, and
the first step includes
a step of allowing the gene analyzer to calculate a scale using the electrophoretic characteristic data associated with the first observation environment and the electrophoretic characteristic data associated with the observation environment different from the first observation environment and
a step of allowing the gene analyzer to scale the mobility correction amount data associated with the observation environment different from the first observation environment based on the scale.

3. The analysis method of a base sequence according to claim 1, wherein
the second step includes
a fourth step of allowing the gene analyzer to generate automatic mobility correction amount data based on the optimization algorithm of the mobility correction amount, and
a fifth step of allowing the gene analyzer to correct the automatic mobility correction amount data based on a difference between the automatic mobility correction amount data and the default mobility correction amount data.

4. The analysis method of a base sequence according to claim 3, wherein
when the overall difference between the automatic mobility correction amount data and the default mobility correction amount data is large, the fifth step includes a step of allowing the gene analyzer to replace the automatic mobility correction amount data by the default mobility correction amount data.

5. The analysis method of a base sequence according to claim 3, wherein
the fifth step includes
a step of allowing the gene analyzer to identify a portion where the difference between the automatic mobility correction amount data and the default mobility correction amount data is large, and
a step of allowing the gene analyzer to replace the identified portion by the default mobility correction amount data.

6. The analysis method of a base sequence according to claim 3, wherein
the fifth step includes
a step of allowing the gene analyzer to identify a portion where the difference between the automatic mobility correction amount data and the default mobility correction amount data is large and a variation is large, and
a step of allowing the gene analyzer to correct the identified portion such that a difference from the default mobility correction amount data decreases.

7. The analysis method of a base sequence according to claim 1, wherein
the third step includes a step of allowing the gene analyzer to store the first observation environment and the used mobility correction amount data in association with each other.

8. The analysis method of a base sequence according to claim 2, further comprising:
a step of allowing the gene analyzer to generate the electrophoretic characteristic data of a plurality of bases in a second observation environment using mobility correction amount data of time-series data of signal intensities of the plurality of bases acquired by electrophoresing the sample in the second observation environment and the base sequence of the sample identified using the corrected time-series data of the signal intensities of the plurality of bases; and
a step of storing the second observation environment and the generated electrophoretic characteristic data in association with each other.

9. A gene analyzer for analyzing a base sequence of a sample using time-series data of signal intensities of a plurality of bases acquired by electrophoresing the sample,
the gene analyzer managing an observation environment and mobility correction amount data for correcting a position in a time direction of the time-series data of the signal intensities of the plurality of bases in association with each other, and executing the following processes including:
a first process of scaling the mobility correction amount data associated with the observation environment different from a first observation environment to generate default mobility correction amount data when the gene analyzer receives time-series data of signal intensities of a plurality of bases acquired by electrophoresing the sample in the first observation environment;
a second process of correcting the position in the time direction of the time-series data of the signal intensities of the plurality of bases using an optimization algorithm of a mobility correction amount and the default mobility correction amount data; and
a third process of identifying the base sequence of the sample using the corrected time-series data of the signal intensities of the plurality of bases.

10. The gene analyzer according to claim 9, wherein
an observation environment and electrophoretic characteristic data representing a relationship between a position of the base depending on electrophoresis and a migration time are managed in association with each other, and
in the first process,
a scale is calculated using the electrophoretic characteristic data associated with the first observation environment and the electrophoretic characteristic data associated with the observation environment different from the first observation environment, and
the mobility correction amount data associated with the observation environment different from the first observation environment is scaled based on the scale.

11. The gene analyzer according to claim 9, wherein
in the second process,
a fourth process of generating automatic mobility correction amount data based on the optimization algorithm of the mobility correction amount, and
a fifth process of correcting the automatic mobility correction amount data based on a difference between the automatic mobility correction amount data and the default mobility correction amount data
are executed.

12. The gene analyzer according to claim 11, wherein
in the fifth process, when the overall difference between the automatic mobility correction amount data and the default mobility correction amount data is large, the automatic mobility correction amount data is replaced by the default mobility correction amount data.

13. The gene analyzer according to claim 11, wherein
in the fifth process,
a portion where the difference between the automatic mobility correction amount data and the default mobility correction amount data is large is identified, and
the identified portion is replaced by the default mobility correction amount data.

14. The gene analyzer according to claim 11, wherein
in the fifth process,
a portion where the difference between the automatic mobility correction amount data and the default mobility correction amount data is large and a variation is large is identified, and
the identified portion is corrected such that a difference from the default mobility correction amount data decreases.

15. An analysis method of a base sequence that is executed by a gene analyzer for analyzing a base sequence of a sample, the analysis method comprising:
a step of allowing the gene analyzer to calculate default mobility correction amount data of a first observation environment when the gene analyzer receives time-series data of signal intensities of a plurality of bases acquired by electrophoresing the sample in the first observation environment;
a step of allowing the gene analyzer to correct a position in a time direction of the time-series data of the signal intensities of the plurality of bases using the default mobility correction amount data; and
a step of allowing the gene analyzer to identify the base sequence of the sample using the corrected time-series data of the signal intensities of the plurality of bases, wherein
the default mobility correction amount data in different observation environments are data capable of being associated with any scale, and
at least a part of the mobility correction amount data used for the correction has change characteristics of a correction amount similar to the default mobility correction amount data.
